(19) United States
(12) Patent Application Publication
(10) Pub. No.: US 2010/0191135 A1
(43) Pub. Date: Jul. 29, 2010

(54) PEN SHAPED ANALYTICAL APPARATUS OF HEART RATE VARIABILITY

(76) Inventors: Bo-Jau KUO, Taipei City (TW); Ching-Hsiu Yang, Taipei City (TW)

Correspondence Address: Muncy, Geissler, Olds & Lowe, PLLC, 4000 Legato Road, Suite 310, FAIRFAX, VA, 22033, US

(21) Appl. No.: 12/360,299

(22) Filed: Jan. 27, 2009

(51) Int. Cl. A61B 5/0404

(52) U.S. Cl. 600/523

(57) ABSTRACT

The present invention provides a pen shaped analytical apparatus of heart rate variability having a view to simplifying the analytical process and carrying out automation. The pen shaped analytical apparatus of heart rate variability mainly includes an electrocardiogram signal converter; a micro-controller unit; a feature extraction unit; a decision making unit; a display unit and a switching unit. It is noted that the pen shaped analytical apparatus of heart rate variability further comprises: a plurality of electrodes and a battery. In practice, the pen shaped analytical apparatus of heart rate variability put forth in the present invention is quite time-saving and easy to use, as it prints out a person heart rate variability analytical result and autonomic function data in just five minutes after a plurality of electrodes are pressed simultaneously.

### BACKGROUND OF THE INVENTION

1. Field of the Invention

The present invention generally relates to a pen shaped analytical apparatus of heart rate variability, and more particularly to programmable and portable pen shaped analytical apparatus of heart rate variability, designed to be used by various users with ease.

2. Description of the Related Art

Sympathetic nerves and parasympathetic nerves, both of which belong to human autonomic nervous system, are closely related to the daily operation of a human body. Autonomic imbalance may induce various acute and chronic diseases, for example, heart disease, hypertension, etc., and may even lead to a sudden death, if serious. In the past, numerous instruments and methods for evaluation of autonomic functions were developed, including heart rate variation with deep breathing, valsalva response, sudomotor function, orthostatic blood pressure recordings, cold pressure test, biochemistry test, etc. However, the abovementioned methods either cause the patients much pain by requiring them to immerse in water during the test, or require expensive instruments. Hence, the above-mentioned methods are not fit to be used widely. In addition, some of these methods are difficult to use because of poor precision.

The sympathetic nerves work slowly, and the parasympathetic nerves (especially the vagus nerve, which controls heart rate) function fast. Mankind has known the discrepancy between the respective speeds of these two different kinds of nervous systems for a long time. However, in the past, the analytical instruments were not sophisticated enough to enable the evaluation of this characteristic or persuade people that it is worth using. The advent of the technology about spectrum analyzers in the early 1980s enabled heart rate variability analysis to be brought into full play, when autonomic functions were quantitatively analyzed in light of the beating cycle of heart. Hence, heart rate variability analysis gradually becomes the best non-invasive method for detecting autonomic functions.

With spectrum analysis, researchers discovered that the minute fluctuations of heart rate variability can be definitely divided into two groups, that is, high-frequency (HF) component and low frequency (LF) component. The HF component is synchronous to animals breath signals, so it is also known as breath component, which occurs approximately every three seconds in a human being. The source of the LF component that takes place approximately every ten seconds in a human being remains unidentified, though researchers infer that they are relevant to vascular motion or baroreflex. Some academics went further to divide the LF component into two categories, that is, very low frequency (VHF) component and low frequency component. At present, many physiologists and cardiologists believe that the HF component or total power (TP) reflects parasympathetic functions, whereas the ratio of LF component to HF component (LF/HF) reflects sympathetic activity.

In 1996, European and American cardiology societies standardized and published the analytical method of heart rate variability (Circulation (1996), 17, pp. 354-381). However, this method is rather complicated and trivial, and researchers have to identify noise, and eliminate them manually, and thus it requires considerable manpower and time to accomplish the chores. Hence, the aforesaid method constitutes a high threshold for laymen to gain access to the method. At present, heart rate variability is mostly analyzed by a digital computer. An electrocardiogram signal is captured and analog-to-digital conversion is performed on it, and then the converted electrocardiogram signal is stored in a digital file. Meanwhile, it is necessary to provide an identification code or a filename for the digital file. Any correction or analysis carried out to the digital file has to be done manually. Upon completion of the analysis, data also has to be printed out manually.

In short, with a conventional method, the process of analysis of heart rate variability, from signal retrieval to file analysis and eventually printout processing, has to be performed manually. In this regard, a keyboard is the usual medium of operation. As a result, the analytical process of heart rate variability involves a lot of keystrokes performed on the part of a researcher and, worse yet, it also involves pressing different types of keys on the keyboard. In addition, equipped with a keyboard, a machine designed to analyze heart rate variability design can never be smaller; this does not conform to the current trend of miniaturization of machines. According the above problems, the related filed need a pen shaped analytical apparatus of heart rate variability to overcome the disadvantage of the prior art. Moreover, U.S. Pat. No. 7,277,746 “Methods and Apparatus for Analyzing Heart Rate Variability” and TW 225394 “Methods and Apparatus for Analyzing Heart Rate Variability” issued to “Kuo, Terry B. J.”, etc. are all cited as the reference in this invention.

### BRIEF SUMMARY OF THE INVENTION

The primary objective of the present invention to provide an apparatus of heart rate variability, with a view to simplifying the analytical process and carrying out automation. Furthermore, the present invention involves filtering out noise by means of statistical method, in order to enhance the precision of the analysis of heart rate variability.

To achieve the above objective, the present invention provides a pen shaped analytical apparatus of heart rate variability, with a view to simplifying the analytical process and carrying out automation. The pen shaped analytical apparatus of heart rate variability comprises an electrocardiogram signal converter; a micro-controller unit; a feature extraction unit; a decision making unit; a display unit and a switching unit. The electrocardiogram signal converter is used for capturing an electrocardiogram signal of a person to proceed heart rate variability. The micro-controller unit is used for processing the heart rate variability of the electrocardiogram signal converter. The feature extraction unit is used for capturing a feature of the heart rate variability from the micro-controller unit and sending it back to the micro-controller unit. The decision making unit is used for deciding the feature of the heart rate variability from the micro-controller unit and sending a deciding result back to the micro-controller unit. The display unit is used for displaying the deciding result of the micro-controller unit. The switching unit is attached on the surface of the pen shaped analytical apparatus of heart rate variability and used for switching the displaying data of the display unit.

According to one aspect of the present invention, the pen shaped analytical apparatus of heart rate variability further comprises: a plurality of electrodes and a battery. The plurality of electrodes is attached on the surface of the pen shaped analytical apparatus of heart rate variability and used for capturing a heartbeat and an electrocardiogram signal of a person. The battery is used for providing the power supply of the pen shaped analytical apparatus of heart rate variability and being one of rechargeable type lithium battery, nickel-metal hydride battery, nickel- cadmium battery cadmium, and one circle battery.

According to another aspect of the present invention, the electrocardiogram signal converter comprises: an electrocardiogram signal detector; a signal amplifier; a filter; an analog-to-digital converter and a digital input/output device. The electrocardiogram signal detector is used for capturing the electrocardiogram signal of a person. The signal amplifier is used for amplifying the electrocardiogram signal. The filter is used for filtering the electrocardiogram signal. The analog-to-digital converter is connected to the signal amplifier for digitizing the electrocardiogram signal. The digital input/output device is connected to the analog-to-digital converter as a communication interface of the electrocardiogram signal.

### BRIEF DESCRIPTION OF THE DRAWINGS

All the objects, advantages, and novel features of the invention will become more apparent from the following detailed descriptions when taken in conjunction with the accompanying drawings.

FIG. 1 shows a schematic of the pen shaped analytical apparatus of heart rate variability according to the present invention;

FIG. 2 shows a process flow chart of the pen shaped analytical apparatus of heart rate variability according to the present invention;

FIG. 3 shows a process flow chart of the electrocardiogram signal converter according to the present invention;

FIG. 4 shows a process flow chart of the feature extraction unit according to the present invention;

FIG. 5 shows a process flow chart of the decision making unit according to the present invention; and

FIG. 6 shows every displaying data of the display unit according to the present invention.

### DETAILED DESCRIPTION OF THE INVENTION

Although the invention has been explained in relation to several preferred embodiments, the accompanying drawings and the following detailed descriptions are the preferred embodiment of the present invention. It is to be understood that the following disclosed descriptions will be examples of present invention, and will not limit the present invention into the drawings and the special embodiment.

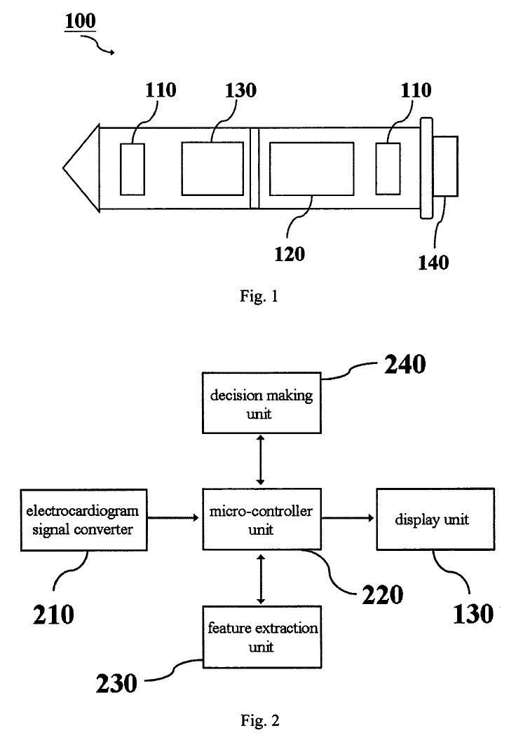

Referring to FIG. 1, it shows a schematic of the pen shaped analytical apparatus of heart rate variability 100 according to the present invention. The pen shaped analytical apparatus of heart rate variability 100 mainly comprises an electrocardiogram signal converter 210; a micro-controller unit 220; a feature extraction unit 230; a decision making unit 240; a display unit 130 and a switching unit 140. The switching unit 140 is attached on the surface of the pen shaped analytical apparatus of heart rate variability 100 and used for switching the displaying data of the display unit 130. It is deserved to be mentioned that the display unit's 130 display result is one of time, frequency domain analysis 232, time domain analysis 231, OK, and Help, and the display unit 130 is one of the liquid crystal display and light emitting diode. The pen shaped analytical apparatus of heart rate variability 100 further comprises: a plurality of electrodes 110 and a battery 120. The plurality of electrodes 110 is attached on the surface of the pen shaped analytical apparatus of heart rate variability 100 and used for capturing a heartbeat and an electrocardiogram signal of a person. The battery 120 is used for providing the power supply of the pen shaped analytical apparatus of heart rate variability 100 and being one of rechargeable type lithium battery 120, nickel-metal hydride battery 120, nickel-cadmium battery 120, and one circle battery 120. Additionally, the plurality of electrodes 110 are one of reactive electrodes 110 and the input electrodes 110 of traditional electro-cardiogram.

Now referring to FIG. 2, it shows a process flow chart of the pen shaped analytical apparatus of heart rate variability 100 according to the present invention.

The electrocardiogram signal converter 210 is used for capturing an electrocardiogram signal of a person to proceed heart rate variability. The micro-controller unit 220 is used for processing the heart rate variability of the electrocardiogram signal converter 210. The feature extraction unit 230 is used for capturing a feature of the heart rate variability from the micro-controller unit 220 and sending it back to the micro-controller unit 220. The decision making unit 240 is used for deciding the feature of the heart rate variability from the micro-controller unit 220 and sending a deciding result back to the micro-controller unit 220. The display unit 130 is used for displaying the deciding result of the micro-controller unit 220.

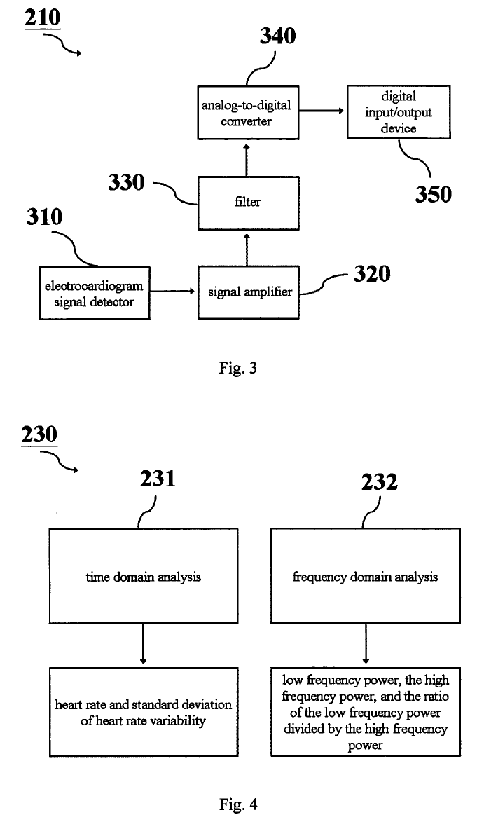

Now referring to FIG. 3, it shows a process flow chart of the electrocardiogram signal converter 210 according to the present invention. U.S. Pat. No. 7,277,746 “Methods and Apparatus for Analyzing Heart Rate Variability” and TW 225394 “Methods and Apparatus for Analyzing Heart Rate Variability” issued to “Kuo, Terry B. J.”, etc. are all cited as the reference in this invention. The electrocardiogram signal converter 210 comprises: an electrocardiogram signal detector 310; a signal amplifier 320; a filter 330; an analog-to-digital converter 340 and a digital input/output device 350. The electrocardiogram signal detector 310 is used for capturing the electrocardiogram signal of a person. The signal amplifier 320 is used for amplifying the electrocardiogram signal. The filter 330 is used for filtering the electrocardiogram signal. It is deserved to be mentioned that the signal amplifier 320 and the filter 330 are one of differential amplifier and single-ended digital amplifier. The analog-to-digital converter 340 is connected to the signal amplifier 320 for digitizing the electrocardiogram signal. The digital input/output device 350 is connected to the analog-to-digital converter 340 as a communication interface of the electrocardiogram signal.

The electrocardiogram signal detector 310 is composed of a plurality of electrodes 110 and attached on the surface of the pen shaped analytical apparatus of heart rate variability 100. One end of each detection electrode is connected to the subject, and the other end passes through the case to be connected to the signal amplifier 320 so as to capture a person electrocardiogram signals and transmit them to the signal amplifier 320. After being amplified by the signal amplifier 320, the electrocardiogram signals are converted into digital signals by means of the analog-to-digital converter 340, and then are entered into the micro-controller unit 220. The micro-controller unit 220 executes a program to carry out a series of analyses and control-related tasks. The digital input/output device 350 functions as the transmission interface between the electrocardiogram signal detector 310 and the subject. In practice, being a user-machine interface intended for external communication, the digital input/output device 350 may be additionally connected to a indicator, to indicate the status of the pen shaped analytical apparatus of heart rate variability 100. Transmission lines connect the signal amplifier 320 and the analog-to-digital converter 340, the analog-to-digital converter 340 and the micro-controller unit 220, and the micro-controller unit 220 and the digital input/output device 350 to transmit signals.

The types of the electronic components of the pen shaped analytical apparatus of heart rate variability 100 which can be implemented include: Bipolar Junction Transistor (BJT), Heterojunction Bipolar Transistor (HBT), High Electronic Mobility Transistor (HEMT), Pseudomorphic HEMT (PHEMT), Complementary Metal Oxide Semiconductor Filed Effect Transistor (CMOS) and Laterally Diffused Metal Oxide Semiconductor Filed Effect Transistor (LDMOS). Semiconductor materials broadly applicable to the electronic components of the pen shaped analytical apparatus of heart rate variability 100 include: silicon, silicon-on-insulator (SOI), silicon-germanium (SiGe), gallium arsenide (GaAs), indium phosphide (InP) and silicon-germanium-carbon (SiGe—C). Preferably, the electrocardiogram signal converter 210 is designed with resistive shunt-feedback PHEMT transistors on semiconductor substrate of Al—In—GaAs compound.

Now referring to FIG. 4, it shows a process flow chart of the feature extraction unit 230 according to the present invention. The feature extraction unit 230 is comprised of a time domain analysis 231 of the heart rate variability and a frequency domain analysis 232 of the heart rate variability. The time domain analysis 231 of the heart rate variability can obtain heart rate and standard deviation of heart rate variability. Additionally, the frequency domain analysis 232 of the heart rate variability can obtain the low frequency power, the high frequency power, and the ratio of the low frequency power divided by the high frequency power. Fourier transform is adopted in the frequency domain analysis 232. In the first place, any linear drift of signal is eliminated to evade the interference from low-frequency band, and the Hamming computation is employed to prevent the mutual leakage between individual frequency components of the spectrum. After that, fast Fourier transform is conducted so as to acquire heart rate power spectral density (HPSD), and the compensation with regard to any effects of sampling and Hamming computation is performed. The low frequency power (0.04-0.15 Hz) and the high frequency power (0.15-0.4 Hz) bands of the heart rate power spectral density are quantified by integral, and the quantitative parameters like the ratio of the low frequency power divided by the high frequency power are captured.

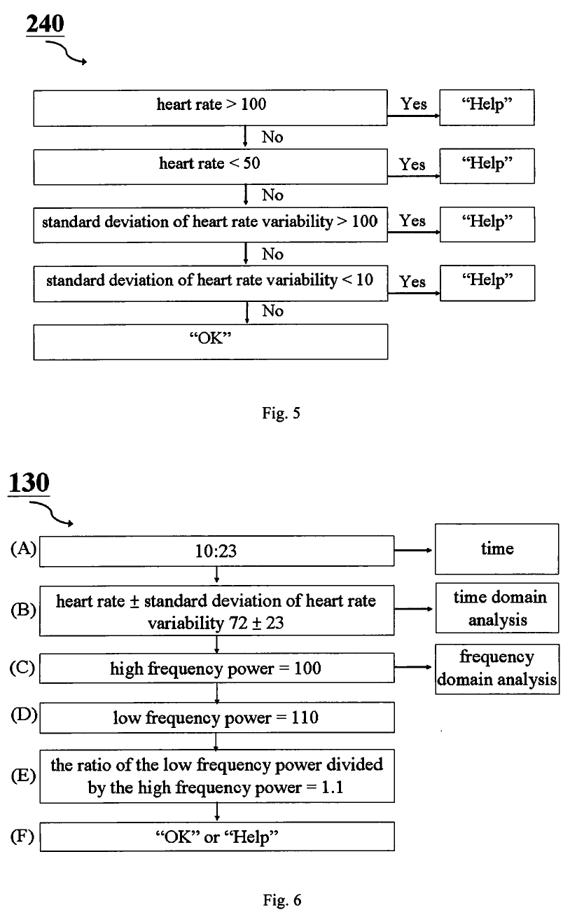

Now referring to FIG. 5, it shows a process flow chart of the decision making unit 240 according to the present invention. The decision making unit 240 sets up a first threshold value and a second threshold value and based on every feature of the heart rate variability from the feature extraction unit 230. When the feature of the heart rate variability of the decision making unit 240 is between the first threshold value and the second threshold value, the decision making unit 240 decides OK. On the contrary, when the feature of the heart rate variability of the decision making unit 240 is higher than the first threshold value or lower than the second threshold value, the decision making unit 240 decides Help. For example, when the heart rate of the decision making unit 240 is between 50 and 100 and the standard deviation of heart rate variability is between 10 and 100, the display unit 130 is displayed OK. On the contrary, when the heart rate of the decision making unit 240 is less than 50 or larger than 100 and the standard deviation of heart rate variability is less than 10 or larger than 100, the display unit 130 is displayed Help.

Now referring to FIG. 6, it shows every displaying data of the display unit according to the present invention: (A) time, (B) heart rate (HR) ± standard deviation of heart rate variability (SD) of the heart rate variability analyzed by the time domain analysis 231, (C) high frequency power (HF) of the heart rate variability analyzed by the frequency domain analysis 232, (D) low frequency power (LF) of the heart rate variability analyzed by the frequency domain analysis 232, (E) the ratio of the low frequency power divided by the high frequency power (LF/HF) of the heart rate variability analyzed by the frequency domain analysis 232, (F) OK or Help.

According to the preferred embodiment of the present invention, the advantage of the pen shaped analytical apparatus of heart rate variability 100 is unlike a conventional heart rate variability analysis that requires a user to enter a large amount of data, the present invention reduces the number of keystrokes to one during the process of heart rate variability analysis, and even the traditional keyboard can be replaced with a plurality of electrodes 110, under the integrated control of the micro-controller unit 220. The method put forth in the present invention may not only be applied to small machines, but also provide a friendly operating interface. Besides tremendously minimizing operational errors, it becomes accessible to laymen. In practice, the pen shaped analytical apparatus of heart rate variability 100 put forth in the present invention is quite time-saving and easy to use, as the display unit 130 displayed a person heart rate variability analytical result and autonomic function data in just five minutes after a plurality of electrodes 110 are pressed simultaneously. Additionally, the dimension of the pen shaped analytical apparatus of heart rate variability 100 is around 13 mm×130 mm, and the process flow chart appeared in the pen shaped analytical apparatus of heart rate variability 100 can be used in the one of cell phone system, personal digital assistant system, 3 C product, watch and temperature.

Although the invention has been explained in relation to its preferred embodiment, it is not used to limit the invention. It is to be understood that many other possible modifications and variations can be made by those skilled in the art without departing from the spirit and scope of the invention as hereinafter claimed.

What is claimed is:

1. A pen shaped analytical apparatus of heart rate variability comprises:
an electrocardiogram signal converter, used for capturing an electrocardiogram signal of a person to proceed heart rate variability;
a micro-controller unit, used for processing the heart rate variability of the electrocardiogram signal converter;
a feature extraction unit, used for capturing a feature of the heart rate variability from the micro-controller unit and sending it back to the micro-controller unit;
a decision making unit, used for deciding the feature of the heart rate variability from the micro-controller unit and sending a deciding result back to the micro-controller unit;
a display unit, used for displaying the deciding result of the micro-controller unit; and
a switching unit, attached on the surface of the pen shaped analytical apparatus of heart rate variability, used for switching the displaying data of the display unit.

2. The pen shaped analytical apparatus of heart rate variability as claimed in claim 1, comprising else:
a plurality of electrodes, attached on the surface of the pen shaped analytical apparatus of heart rate variability, used for capturing a heartbeat and an electrocardiogram signal of a person; and
a battery, used for providing the power supply of the pen shaped analytical apparatus of heart rate variability, being one of rechargeable type lithium battery, nickel-metal hydride battery, nickel-cadmium battery cadmium, and one circle battery.

3. The pen shaped analytical apparatus of heart rate variability as claimed in claim 2, wherein the plurality of electrodes are one of reactive electrodes and the input electrodes of traditional electro-cardiogram.

4. The pen shaped analytical apparatus of heart rate variability as claimed in claim 1, wherein the electrocardiogram signal converter comprises:
an electrocardiogram signal detector, used for capturing the electrocardiogram signal of a person;
a signal amplifier, used for amplifying the electrocardiogram signal;
a filter, used for filtering the electrocardiogram signal;
an analog-to-digital converter, connected to the signal amplifier for digitizing the electrocardiogram signal; and
a digital input/output device, connected to the analog-to-digital converter as a communication interface of the electrocardiogram signal.

5. The pen shaped analytical apparatus of heart rate variability as claimed in claim 4, wherein the signal amplifier and the filter are one of differential amplifier and single-ended digital amplifier.

6. The pen shaped analytical apparatus of heart rate variability as claimed in claim 1, wherein the feature extraction unit comprises of a time domain analysis of the heart rate variability and a frequency domain analysis of the heart rate variability.

7. The pen shaped analytical apparatus of heart rate variability as claimed in claim 6, wherein the time domain analysis of the heart rate variability can obtain heart rate and standard deviation of heart rate variability.

8. The pen shaped analytical apparatus of heart rate variability as claimed in claim 6, wherein the frequency domain analysis of the heart rate variability can obtain the low frequency power, the high frequency power, and the ratio of the low frequency power divided by the high frequency power.

9. The pen shaped analytical apparatus of heart rate variability as claimed in claim 1, wherein the display unit's display result is one of time, frequency domain analysis, time domain analysis, OK, and Help.

10. The pen shaped analytical apparatus of heart rate variability as claimed in claim 6, wherein the decision making unit sets up a first threshold value and a second threshold value, based on every feature of the heart rate variability from the feature extraction unit.

11. The pen shaped analytical apparatus of heart rate variability as claimed in claim 10, wherein the decision making unit decides OK, when the feature of the heart rate variability of the decision making unit is between the first threshold value and the second threshold value.

12. The pen shaped analytical apparatus of heart rate variability as claimed in claim 10, wherein the decision making unit decides Help, when the feature of the heart rate variability of the decision making unit is higher than the first threshold value or lower than the second threshold value.

13. The pen shaped analytical apparatus of heart rate variability as claimed in claim 1, wherein the display unit is one of the liquid crystal display and light emitting diode.

14. The pen shaped analytical apparatus of heart rate variability as claimed in claim 1, wherein the dimension of the pen shaped analytical apparatus of heart rate variability is around 13 mm×130 mm.

15. The pen shaped analytical apparatus of heart rate variability as claimed in claim 1, wherein the process flow chart appeared in the pen shaped analytical apparatus of heart rate variability can be used in the one of cell phone system, personal digital assistant system, 3 C product, watch and temperature.
